(19) United States
(12) Patent Application Publication
(10) Pub. No.: US 2015/0173738 A1
(43) Pub. Date: Jun. 25, 2015

(54) SURGICAL FIXATION DEVICE WITH ANTIBACTERIAL SUBSTANCE

(71) Applicant: Arthrex, Inc., Naples, FL (US)

(72) Inventor: John W. Schmieding, Naples, FL (US)

(21) Appl. No.: 14/525,696

(22) Filed: Oct. 28, 2014

Related U.S. Application Data
(60) Provisional application No. 61/920,004, filed on Dec. 23, 2013.

(51) Int. Cl. A61B 17/04; A61F 2/08; A61B 17/06

(52) U.S. Cl. CPC A61B 17/0401; A61B 17/06166; A61F 2/0811; A61B 2017/0464; A61B 2017/00889

(57) ABSTRACT

A method according to an exemplary aspect of the present disclosure includes, among other things, forming a passage in a bone and positioning a surgical fixation device that includes an antibacterial substance at least partially in the passage. The antibacterial substance inhibits the spread of infection through the passage.

### CROSS REFERENCE TO RELATED APPLICATION

This application claims priority to U.S. Provisional Application No. 61/920,004, which was filed on Dec. 23, 2013.

### BACKGROUND

This disclosure relates to a surgical device and method of using the same to inhibit the spread of infection. More particularly, this disclosure is directed to a surgical fixation device that includes an antibacterial substance.

Orthopedic procedures are often performed to repair musculoskeletal injuries. For example, soft tissue may tear away from bone during vigorous exercise or sporting activities. When tears occur, reattachment is often necessary to repair the damaged tissue.

Various surgical fixation devices including but not limited to sutures, screws, staples, wedges, buttons, plugs and anchors are currently used to repair damaged tissue Implantation of these types of devices typically requires breaching the skin and other body tissue via one or more incisions to reach the joint under need of repair. Additional procedures (i.e., drilling of bone, etc.) may also be required to complete the surgical procedure. Disturbing the skin, body tissue and/or bone in this manner may create an opportunity for bacteria and other microbes to enter into the body.

### SUMMARY

A method according to an exemplary aspect of the present disclosure includes, among other things, forming a passage in a bone and positioning a surgical fixation device that includes an antibacterial substance at least partially in the passage. The antibacterial substance inhibits the spread of infection through the passage.

In a further non-limiting embodiment of the foregoing method, the surgical fixation device is a suture.

In a further non-limiting embodiment of either of the foregoing methods, the antibacterial substance includes a silver ion.

In a further non-limiting embodiment of any of the foregoing methods, the surgical fixation device is coated with the antibacterial substance.

In a further non-limiting embodiment of any of the foregoing methods, the surgical fixation device is impregnated with the antibacterial substance.

In a further non-limiting embodiment of any of the foregoing methods, the surgical fixation device is a suture that includes a jacket and a core surrounded by the jacket, the jacket including a plurality of braided strands, and the antibacterial substance is applied to the plurality of braided strands.

In a further non-limiting embodiment of any of the foregoing methods, the surgical fixation device is a suture anchor.

In a further non-limiting embodiment of any of the foregoing methods, the surgical fixation device is an interference screw.

In a further non-limiting embodiment of any of the foregoing methods, the forming step includes forming a first opening through a first outer surface of the bone and a second opening through a second outer surface of the bone.

In a further non-limiting embodiment of any of the foregoing methods, the method includes fixating the surgical fixation device to the bone with a second surgical fixation device.

In a further non-limiting embodiment of any of the foregoing methods, the second surgical fixation device includes an antibacterial substance.

In a further non-limiting embodiment of any of the foregoing methods, the positioning step includes positioning a portion of the surgical fixation device near an opening of the passage to create a barrier to the spread of infection through the passage.

In a further non-limiting embodiment of any of the foregoing methods, the barrier blocks the spread of infection from an opening of the passage toward an interior portion of the passage.

In a further non-limiting embodiment of any of the foregoing methods, the barrier blocks the spread of infection from the opening toward a second opening of the passage.

In a further non-limiting embodiment of any of the foregoing methods, the bone is a femur and the passage extends entirely through the femur.

A method according to another exemplary aspect of the present disclosure includes, among other things, positioning a surgical fixation device that includes an antibacterial substance within a passage of a bone, the passage defining a conduit through the bone. The antibacterial substance inhibits the spread of infection through the conduit to an internal joint space.

The embodiments, examples and alternatives of the preceding paragraphs, the claims, or the following description and drawings, including any of their various aspects or respective individual features, may be taken independently or in any combination. Features described in connection with one embodiment are applicable to all embodiments, unless such features are incompatible.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 illustrates a surgical fixation device.

FIG. 2 illustrates a cross-sectional view of the surgical fixation device of FIG. 1.

FIG. 3 illustrates another surgical fixation device.

FIG. 4 illustrates yet another surgical fixation device.

FIG. 5 schematically illustrates a method of creating a barrier to the spread of infection within a joint.

FIG. 6 schematically illustrates a method for blocking the spread of infection through a bone passage.

### DETAILED DESCRIPTION

This disclosure relates to a surgical fixation device that includes an antibacterial substance. The antibacterial substance can control or limit the development and/or spread of infection within a body. The surgical fixation device may be coated, impregnated or otherwise provided with the antibacterial substance and subsequently utilized in a surgical procedure to block the spread of infection through a passage formed in a bone. These and other features are discussed in greater detail in the following paragraphs.

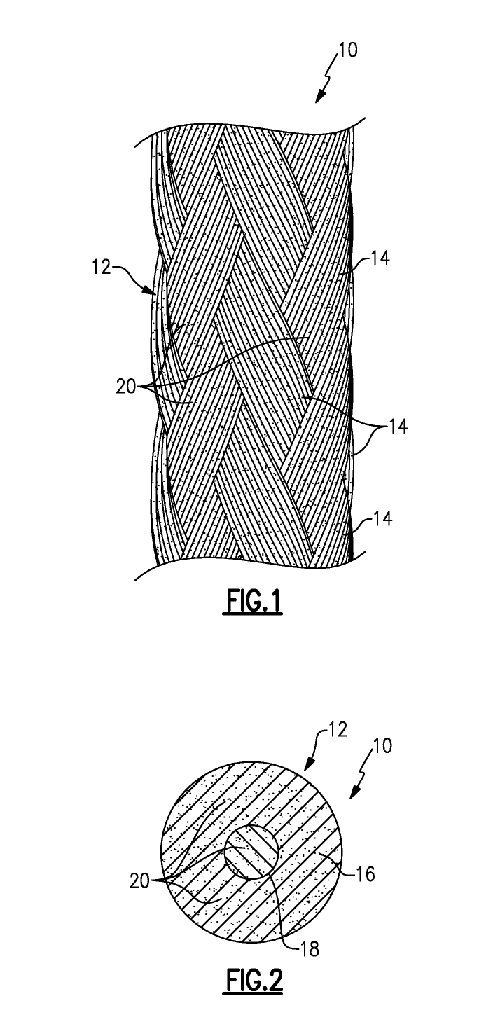

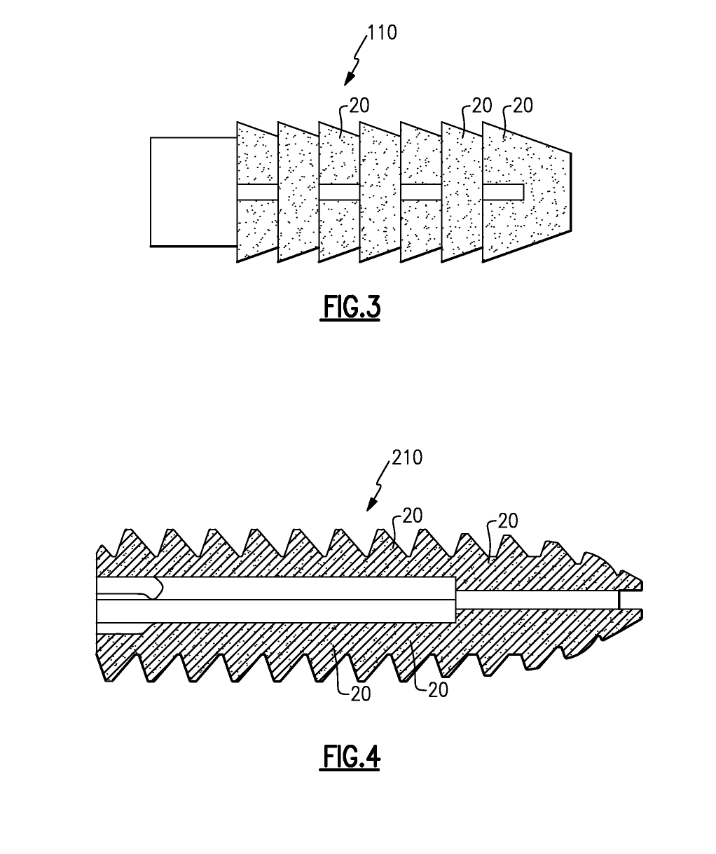

FIGS. 1 and 2 illustrate a surgical fixation device 10. In this embodiment, the surgical fixation device 10 is a section of suture 12 that may be formed of one or more strands 14 of a flexible material(s). It should be appreciated that FIGS. 1 and 2 have been enlarged to better illustrate the features of the surgical fixation device 10.

In one embodiment, the suture 12 includes a jacket 16 and a core 18 surrounded by the jacket 16. The strands 14 may be braided together to form the jacket 16. The core 18 may be similarly formed by twisting strands of a material that can be similar or dissimilar from the material of the jacket 16. Alternatively, the suture 12 could be a monofilament suture that does not include a core.

In one embodiment, the suture 12, including the jacket 16 and the core 18, is formed from a high strength suture material. One non-limiting example of such a high strength suture material is illustrated by Arthrex's Fiberwire® suture, which is described in U.S. Pat. No. 6,716,234, the disclosure of which is incorporated herein by reference. Fiberwire suture may be formed of braided strands 14 of ultra-high molecular weight polyethylene (UHMWPE) and polyester, in one embodiment.

In another non-limiting embodiment, the suture 12 is formed of a plurality of braided strands 14 of a polyether-ketone (PEEK) variant. One such suture is described in U.S. patent application Ser. No. 11/775,079, the disclosure of which is incorporated herein by reference. Other materials may also be suitable for constructing the suture 12.

The surgical fixation device 10 may be provided with an antibacterial substance 20. The antibacterial substance 20 can inhibit the development and spread of infection within a body. The antibacterial substance 20 may be applied locally to portions of the surgical fixation device 10 or could be applied to the entire surface area of the surgical fixation device 10.

In one embodiment, the strands 14 of the suture 12 are coated with the antibacterial substance 20. In another embodiment, the strands 14 are impregnated with the antibacterial substance 20. The antibacterial substance 20 could alternatively be applied to the surgical fixation device 10 by soaking, injection or any other known manner.

In one embodiment, the antibacterial substance 20 includes a silver ion. However, other antibacterial substances are additionally contemplated as within the scope of this disclosure. Non-limiting examples of suitable antibacterial substances include antiseptic agents, tetracycline hydrochloride, neomycin sulfate, chloramphenicol, streptomycin sulfate, potassium penicillin, polymixin B sulfate, oxytetracycline hydrochloride, gentamycin sulfate, sodium cephalothin, nitrofurazone, rifamycin, benzethonium chloride, sodium oxacillin, dihydrostreptomycin sulfate, disodium carbenicillin, and sodium furadantin.

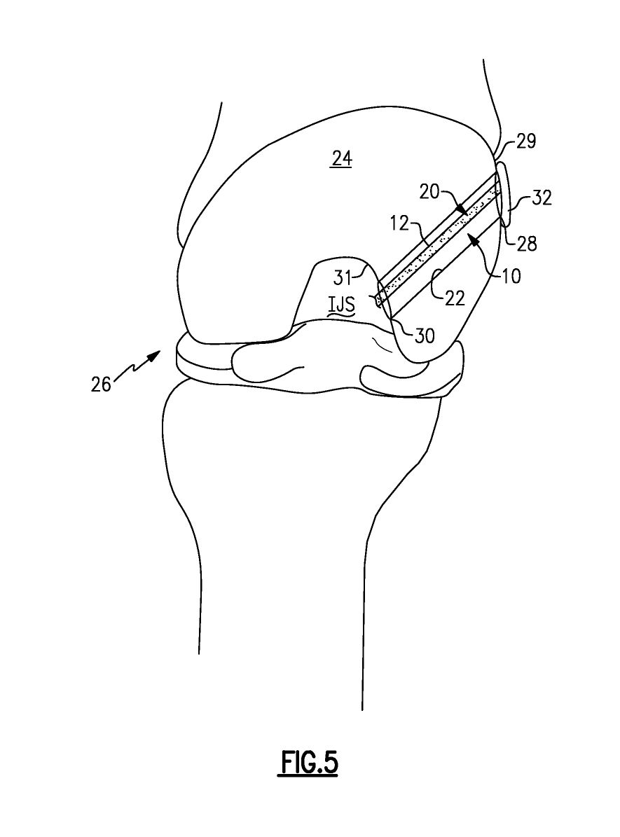

Other surgical fixation devices could benefit from the use of the antibacterial substance 20 described above. For example, FIG. 3 illustrates a suture anchor 110 that includes the antibacterial substance 20. FIG. 4 illustrates an interference screw 210 that includes the antibacterial substance 20. These Figures represent but two additional examples of surgical fixation devices that may be coated, impregnated or otherwise supplied with the antibacterial substance 20 for use in inhibiting the spread of infection within a body.

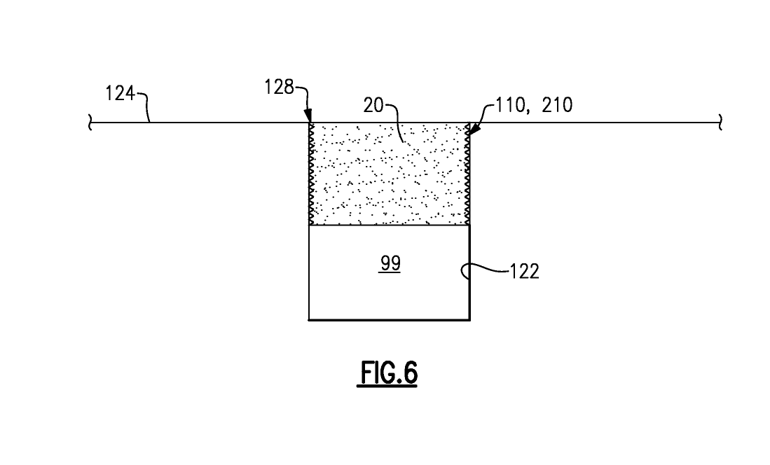

FIG. 5 schematically illustrates a method for blocking or inhibiting the spread of infection within a body, such as a human or animal body. The method may employ the surgical fixation device 10 of FIG. 1, including the suture 12 having the antibacterial substance 20. The suture 12 is used for exemplary purposes only to describe the method of FIG. 5. It should be understood that the surgical fixation devices of FIGS. 3 and 4, or any other surgical fixation devices, could be used in a similar method within the scope of this disclosure.

A passage 22 is formed in a bone 24 of a joint 26. In one non-limiting embodiment, the joint 26 is a knee joint and the bone 24 is a femur of the human body. However, the technique described with respect to FIG. 5 could be utilized in other joints within the scope of this disclosure. The passage 22 may be formed using known techniques and known instrumentation.

The passage 22 is created such that it extends entirely through the bone 24. The passage 22 may include a first opening 28 that exits through a first outer surface 29 of the bone 24 and a second opening 30 that exits through a second, different outer surface 31 of the bone 24.

In some circumstances, the passage 22 may define a conduit for the spread of infection during or subsequent to a surgical procedure. The method of FIG. 5 is designed to reduce the likelihood of an infection spreading through the passage 22.

The surgical fixation device 10 may be at least partially positioned within the passage 22. For example, in one non-limiting embodiment, a suture 12 impregnated with the antibacterial substance 20 is positioned such that at least a portion of the suture 12 that includes the antibacterial substance 20 is within the passage 22. The suture 12 may extend partially or entirely across the passage 22.

In one embodiment, the surgical fixation device 10 could be fixated relative to the bone 24 with a second surgical fixation device 32. For example, the suture 12 may be affixed at a desired positioning relative to the passage 22 using a button (shown), a screw, an anchor or any other surgical fixation device. The second surgical fixation device 32 may or may not be impregnated or coated with the antibacterial substance 20. This may be determined on a case-by-case basis and may depend on the amount of infection control deemed necessary, among other factors.

Positioning the surgical fixation device 10 within the passage 22, such as near at least one of the first and second openings 28, 30, may prevent the spread of infection through the passage 22 from the first opening 28 toward the opposite second opening 30 (or vice-versa). In other words, the surgical fixation device 10 with the antibacterial substance 20 creates a barrier to the spread of infection to an internal join space US of the joint 26 through the passage 22, thereby improving surgical outcomes.

The method schematically illustrated in FIG. 5 is but one exemplary technique for using the surgical fixation devices described by this disclosure. Additional surgical applications may be possible that utilize a surgical fixation device that is provided with an antibacterial substance.

For example, FIG. 6 schematically illustrates another method for blocking or inhibiting the spread of infection within a body. The method of FIG. 6 may employ the surgical fixation device 110 of FIG. 3 (i.e., a suture anchor) or the surgical fixation device 210 of FIG. 4 (i.e., an interference screw). The surgical fixation device 110, 210 is provided with the antibacterial substance 20 and may be inserted into a passage 122 formed in a bone 124. The passage 122 includes an opening 128. The surgical fixation device 110, 210 is fixated near the opening 128 to block the spread of infection further into the passage 122. In one embodiment, the antibacterial substance 20 blocks the spread of infection from the opening 128 toward an interior portion 99 of the passage 124.

Although the different non-limiting embodiments are illustrated as having specific components, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should also be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. A method, comprising:
forming a passage in a bone; and
positioning a surgical fixation device that includes an antibacterial substance at least partially in the passage, wherein the antibacterial substance inhibits the spread of infection through the passage.

2. The method as recited in claim 1, wherein the surgical fixation device is a suture.

3. The method as recited in claim 1, wherein the antibacterial substance includes a silver ion.

4. The method as recited in claim 1, wherein the surgical fixation device is coated with the antibacterial substance.

5. The method as recited in claim 1, wherein the surgical fixation device is impregnated with the antibacterial substance.

6. The method as recited in claim 1, wherein the surgical fixation device is a suture that includes a jacket and a core surrounded by the jacket, the jacket including a plurality of braided strands, and the antibacterial substance is applied to the plurality of braided strands.

7. The method as recited in claim 1, wherein the surgical fixation device is a suture anchor.

8. The method as recited in claim 1, wherein the surgical fixation device is an interference screw.

9. The method as recited in claim 1, wherein the forming step includes forming a first opening through a first outer surface of the bone and a second opening through a second outer surface of the bone.

10. The method as recited in claim 1, comprising fixating the surgical fixation device to the bone with a second surgical fixation device.

11. The method as recited in claim 10, wherein the second surgical fixation device includes an antibacterial substance.

12. The method as recited in claim 1, wherein the positioning step includes positioning a portion of the surgical fixation device near an opening of the passage to create a barrier to the spread of infection through the passage.

13. The method as recited in claim 12, wherein the barrier blocks the spread of infection from an opening of the passage toward an interior portion of the passage.

14. The method as recited in claim 12, wherein the barrier blocks the spread of infection from the opening toward a second opening of the passage.

15. The method as recited in claim 1, wherein the bone is a femur and the passage extends entirely through the femur.

16. The method as recited in claim 1, wherein the antibacterial substance blocks the spread of infection through the passage to an internal joint space.

17. A method, comprising:
positioning a surgical fixation device that includes an antibacterial substance within a passage of a bone, the passage defining a conduit through the bone, wherein the antibacterial substance inhibits the spread of infection through the conduit to an internal joint space.
